(19) United States
(12) Patent Application Publication
(10) Pub. No.: US 2014/0228829 A1
(43) Pub. Date: Aug. 14, 2014

(54) LASER-BASED DEVICES AND METHODS FOR RENAL DENERVATION

(71) Applicant: ST. JUDE MEDICAL, CARDIOLOGY DIVISION, INC., St. Paul, MN (US)

(72) Inventors: Joseph Michael Schmitt, Andover, MA (US); Chengyang Xu, Devens, MA (US); Desmond Adler, Billerica, MA (US)

(73) Assignee: ST. JUDE MEDICAL, CARDIOLOGY DIVISION, INC., St. Paul, MN (US)

(21) Appl. No.: 13/766,040

(22) Filed: Feb. 13, 2013

(51) Int. Cl. A61B 18/24

(52) U.S. Cl. CPC A61B 18/24
USPC 606/10; 606/15

(57) ABSTRACT

An ablation catheter includes an elongated sheath configured for intravascular usage, and an inner tube disposed within the elongated sheath. The inner tube is rotatable and translatable relative to the sheath. An optical fiber is disposed within the inner tube and extends longitudinally therethrough. A proximal end of the optical fiber is optically coupled to a light source and a distal end of the optical fiber is connected to a beam director configured to focus energy on target tissue inside a blood vessel to ablate the target tissue.

### BACKGROUND OF THE INVENTION

The present invention is related to ablation devices, and more particularly to devices, systems, and methods for laser-based ablation for renal denervation.

Hypertension is a major global public health concern. An estimated 30-40% of the adult population in the developed world suffers from this condition. Furthermore, its prevalence is expected to increase, especially in developing countries. Diagnosis and treatment of hypertension remain suboptimal, even in developed countries. Despite the availability of numerous safe and effective pharmacological therapies, including fixed-drug combinations, the percentage of patients achieving adequate blood-pressure control to guideline target values remains low. Much of the failure of the pharmacological strategy to attain adequate blood-pressure control is attributable to both physician inertia and patient non-compliance and non-adherence to a lifelong pharmacological therapy for a mainly asymptomatic disease. Thus, the development of new approaches for the management of hypertension is a priority. These considerations are especially relevant to patients with so-called resistant hypertension (i.e., those unable to achieve target blood-pressure values despite multiple drug therapies at the highest tolerated dose). Such patients are at high risk of major cardiovascular events.

Renal sympathetic efferent and afferent nerves, which lie within and immediately adjacent to the wall of the renal artery, are crucial for initiation and maintenance of systemic hypertension. Indeed, sympathetic nerve modulation as a therapeutic strategy in hypertension had been considered long before the advent of modern pharmacological therapies. Radical surgical methods for thoracic, abdominal, or pelvic sympathetic denervation had been successful in lowering blood pressure in patients with so-called malignant hypertension. However, these methods were associated with high perioperative morbidity and mortality and long-term complications, including bowel, bladder, and erectile dysfunction, in addition to severe postural hypotension. Renal denervation is the application of a chemical agent, or a surgical procedure, or the application of energy to partially or completely damage renal nerves so as to partially or completely block the renal nerve activities. Renal denervation reduces or completely blocks renal sympathetic nerve activity, increases renal blood flow (RBF), and decreases renal plasma norepinephrine (NE) content.

The objective of renal denervation is to neutralize the effect of renal sympathetic system which is involved in arterial hypertension. One method to achieve this objective is to use radio frequency (RF) ablation of renal sympathetic nerves to reduce the blood pressure of certain patients. In preliminary studies, RF ablation of the efferent sympathetic nerves to the kidneys has been shown to produce consistent blood pressure reduction with minimal procedural risk and long-term side effects.

Other techniques may be available to ablate the renal sympathetic nerves. Preferably, such techniques would limit vessel damage, vessel perforation, and generation of thrombus while effectively ablating the tissue. In addition, such techniques may provide feedback methods for effective therapy.

Thus, there is a need for devices and techniques that are designed to minimize certain risks while effectively ablating tissue.

### BRIEF SUMMARY OF THE INVENTION

To achieve these goals, novel laser-based devices are disclosed to enable proper therapy. Certain configurations of laser delivery catheters as well as associated parameters, such as beam width and wavelengths are disclosed to control the selectivity of nerve heating and speed and safety of a renal denervation procedure.

In some embodiments, an ablation catheter includes an elongated sheath configured for intravascular usage and an inner tube disposed within the elongated sheath. The inner tube may be rotatable and translatable relative to the sheath. An optical fiber having a proximal end and a distal end is disposed within the inner tube and extends longitudinally therethrough, the proximal end of the optical fiber being optically coupleable to a light source. A beam director may be coupled to the distal end of the optical fiber and configured to focus energy from the light source on target tissue inside a blood vessel to ablate the target tissue.

In some examples, the light source may be selected from the group consisting of a diode laser and a doped fiber laser pumped with a diode laser. A reflector may be disposed within the beam director and configured to focus the energy from the light source on the target tissue. The light source may emit light at a wavelength of between about 950 nm and about 1000 nm. A controller may be configured to control the emission of light from the light source for a duration of between about 2 seconds and about 20 seconds. A plurality of irrigation fluid channels may be disposed on the beam director and configured to direct irrigation fluid toward the target tissue.

In some examples, a centering balloon may be coupled to an inflation shaft, the centering balloon being configured to properly position the ablation catheter within the blood vessel when an inflation medium is introduced through the inflation shaft to inflate the centering balloon. The centering balloon may be disposed about the beam director. The centering balloon may be elongated to allow the beam director to form discrete lesions at multiple longitudinal levels along the blood vessel within the centering balloon. The centering balloon may include at least one channel extending from a first longitudinal end of the balloon to a second longitudinal end of the balloon to allow blood to continuously pass through the blood vessel while the catheter is disposed therein. The centering balloon may include at least one perforation to allow the inflation medium such as saline to pass therethrough into the blood vessel.

In some examples, a plurality of centering balloons may be coupled to an inflation shaft, the plurality of centering balloons being configured to properly position the ablation catheter within a blood vessel when an inflation medium is introduced through the inflation shaft to inflate the centering balloons. A faceted reflector may be disposed within the beam director and configured to focus the energy from the light source in at least two different radial directions. A detector may be disposed within the beam director and configured to provide feedback of energy reflected from the target tissue.

In some embodiments, an ablation catheter may include an elongated sheath, a plurality of tubes disposed within the elongated sheath and translatable relative to the elongated sheath. The plurality of tubes may be resiliently biased outwardly away from the elongated sheath. An optical fiber may be disposed within each of the tubes, each of the optical fibers having a proximal end and a distal end, the proximal end of each of the optical fibers being optically coupleable to a light source. A beam director may be coupled to the distal end of each of the optical fibers and a distal expander coupled to the plurality of tubes and configured to focus energy from the light source on target tissue of a blood vessel to ablate the target tissue.

In some examples, the plurality of tubes may form a collapsible basket-like arrangement. The plurality of tubes may be formed of nitinol. The plurality of tubes may include four tubes arranged circumferentially apart by 90 degrees. The optical fibers in each of the plurality of tubes may deliver energy of the same wavelength.

### BRIEF DESCRIPTION OF THE DRAWINGS

Various embodiments of the present system and method will now be discussed with reference to the appended drawings. It is to be appreciated that these drawings depict only some embodiments and are therefore not to be considered as limiting the scope of the present system and method.

FIG. 1A is a schematic illustration of a kidney, the renal artery and the aorta;

FIG. 1B is a schematic representation showing an exemplary histological cross-section of a renal artery and the associated density of renal nerves;

FIG. 1C is a plot of optical absorbers in tissue at differing wavelengths;

FIG. 2A is a side perspective view of a laser ablation system in accordance with a first embodiment of the present invention;

FIG. 2B is an enlarged top view of the laser ablation system of FIG. 2A;

FIG. 2C is an enlarged side view of the laser ablation system of FIG. 2A;

FIG. 2D is a side perspective view of the laser ablation system of FIG. 2A in the retracted position;

FIG. 3A is a side view of a laser ablation system in accordance with a second embodiment of the present invention;

FIG. 3B is a side view of the laser ablation system of FIG. 3A in the retracted position;

FIG. 3C is a side view of a variation of the laser ablation system shown in FIG. 3A;

FIG. 4A is a side perspective view of a laser ablation system in accordance with a third embodiment of the present invention;

FIG. 4B is a schematic representation of a faceted reflector forming elliptical spots;

FIG. 5 is a side perspective view of a laser ablation system in accordance with a fourth embodiment of the present invention;

FIG. 6A is a schematic cross-sectional view of a balloon having a channel in accordance with one embodiment of the present invention;

FIG. 6B is a schematic cross-sectional view of a balloon having a channel in accordance with a second embodiment of the present invention;

FIG. 7A is a schematic representation of an optical spectrum feedback system; and

FIG. 7B is a plot of optical absorbers showing the absorption bands of blood-perfused tissue.

### DETAILED DESCRIPTION

In the description that follows, the terms “proximal” and “distal” are to be taken as relative to a user (e.g., a surgeon or a physician) of the disclosed devices and methods. Accordingly, “proximal” is to be understood as relatively close to the user, and “distal” is to be understood as relatively farther away from the user.

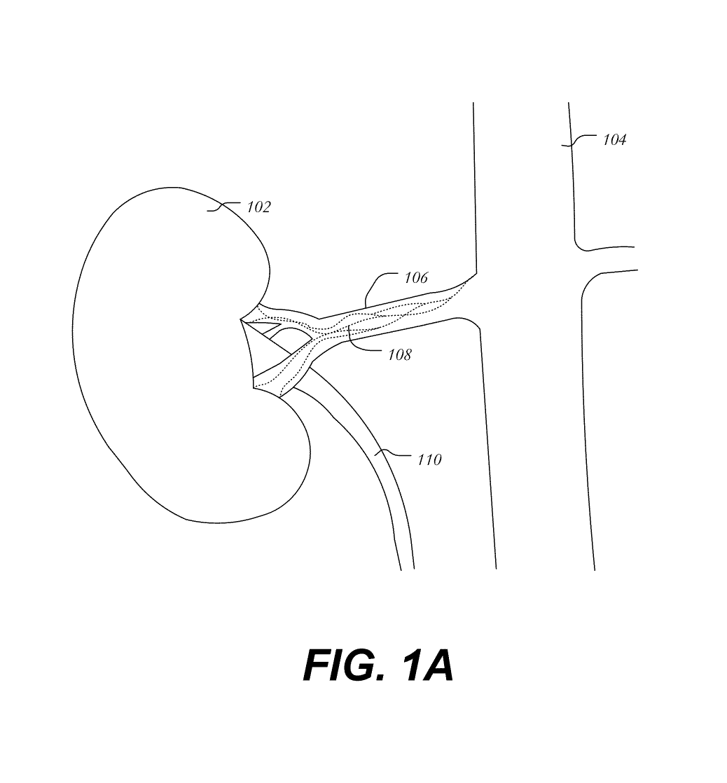

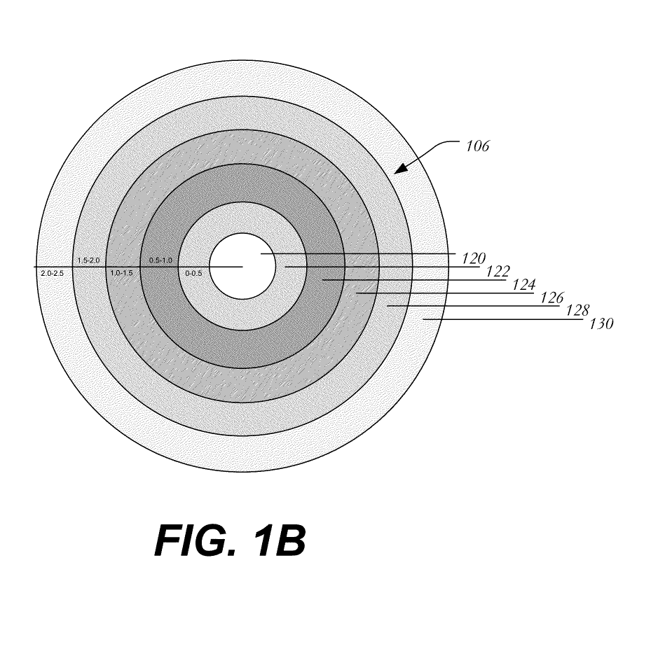

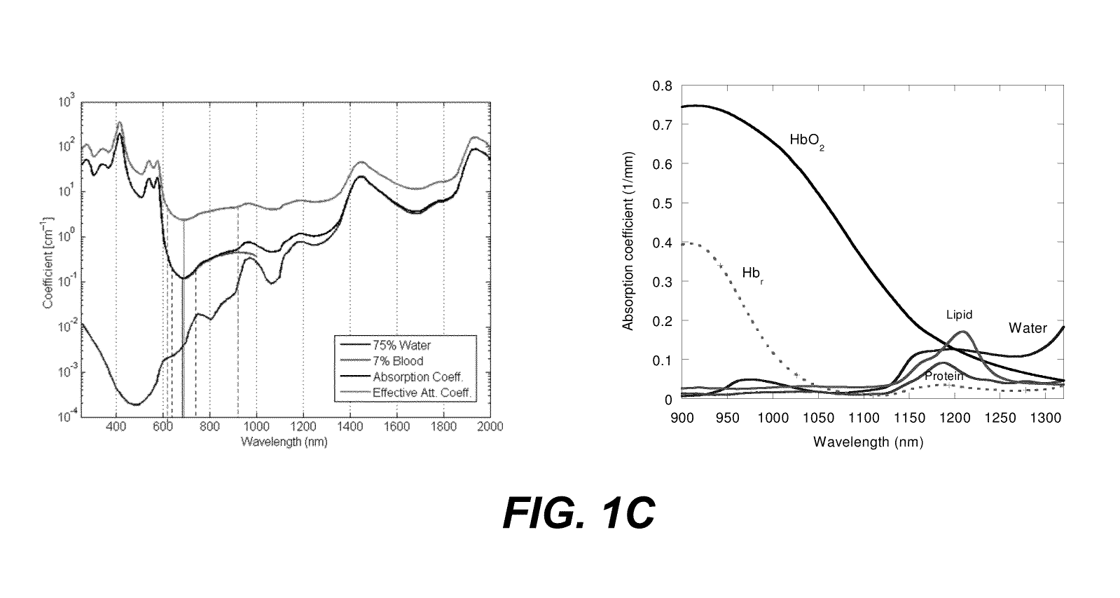

FIG. 1A is a schematic representation of a kidney and its associated structures. The human body typically includes two kidneys 102, one on each side of the vertebral column. The kidneys serve to filter waste products from the blood. After filtration, urine passes from each bean-shaped kidney 102 via ureter 110 to the bladder (not shown). As seen in FIG. 1A, kidneys 102 receive blood from aorta 104 through renal artery 106. Though the main function of the kidneys 102 is to remove waste products from the body, they also play a role as a regulatory organ. Specifically, it has been determined that renal sympathetic efferent and afferent nerves 108, which lie within and adjacent to the wall of the renal artery 106, play a role in managing blood pressure.

Elevated renal nerve activity is associated with the development of essential hypertension. FIG. 1B is a schematic exemplary representation showing a histological cross-section of a renal artery and the associated density of renal nerves from a human cadaver. Renal artery 106 has a lumen 120 through which blood travels. Ring 122 represents a 0.5 mm radius from the outer boundary of lumen 120 and each subsequent ring 124, 126, 128, 130 represents a 0.5 mm radius from the preceding ring. The shading of FIG. 1B illustrates the density of efferent renal sympathetic nerves around the circumference of renal artery 106, with a darker shading representing a more dense distribution of renal nerves 108 than lighter shadings. As seen in FIG. 1B, this model shows that a large portion of the renal nerves are typically distributed 0.5 to 2.5 mm from the outer boundary of lumen 120.

Renal nerves may be permanently destroyed at sustained temperatures greater than 45 degrees Celsius. Cell death may occur within a few seconds at temperatures above 60 degrees Celsius. Adverse effects can occur if the temperature is raised too high: blood may coagulate, the water inside the tissue may vaporize to form a pocket of gas that can release suddenly (steam pop) and cause vessel damage or perforation, and dehydrated tissue at the surface may be charred.

Laser ablation techniques may be effective for several reasons. First, energy deposition depth may be controlled with laser ablation because the beam area and wavelength may be designed to achieve a desired temperature profile. Second, laser ablation may provide suitable guidance and feedback because it is possible to perform optical and spectroscopic measurements indicative of tissue contact and water and blood content. Third, delivery may well-controlled and utilize low power. Finally, laser ablation may result in lower ablation times because wavelength, power and beam width may be adjusted to minimize the gradient of temperature with depth at the ablation site.

Laser ablation relies on the laser heating of tissue absorbers. Specifically, the temperature distribution inside tissue is the net result of three basic processes: (a) the volume heating of the tissue by absorption of the incident laser beam as it scatters through the tissue, (b) heat conduction, convection, and diffusion at the ablation site, and (c) the cooling effect from the blood, irrigation or device components (e.g., catheter body or balloon).

In the ultraviolet and visible wavelength bands (e.g., 300-800 nm), the main sources of optical absorption in biological tissue are hemoglobin, bilirubin and the mitochondrial cytochromes (FIG. 1C). Above 800 nm, in the near-infrared and infrared bands, water, protein, carbohydrates and lipids are the main origin of optical absorption. Specifically, hemoglobin absorption dominates below approximately 1000 nm, and water and lipid absorption dominate above 1000 nm. Moreover, 1060 nm has been found to be a particularly desirable wavelength for suitable penetration.

As for the second two processes of heat conduction and diffusion and the cooling effects from the blood, irrigation or device components, the effects from laser ablation are similar to those of radio frequency ablation. Regarding the volume heating of the tissue, the mechanism of laser-based ablation may be thus summarized: the degree of tissue heating is proportional to the product of tissue absorption and the photon density distribution. For a wide collimated or uniformly diffused beam, the photon density distribution P(z) in the center of the beam may be expressed as an exponentially decaying function where the exponent is determined by the effective attenuation coefficient μ_{eff}˜exp(−sqrt(3μₐμₛ′z), where μₐ is the absorption coefficient and μₛ is the transport-corrected scattering coefficient. These relationships may be summarized by the following equations:

Heat generation=P(z)*μₐ

P(z)=A₀exp(−z*μ_{eff})

It has been found that in wavelength bands of interest, 0.5≦μₛ≦1.5 mm⁻¹ and 0.01≦μₐ≦0.5 mm⁻¹ for arterial tissue and periadventitial tissue surrounding blood vessels. Thus, the wavelength band 1050-1100 nm would likely yield the greatest penetration depth and widest heating zone, without much selective blood absorption. For example, at 1064 nm, the effective penetration depth (the inverse of effective attenuation coefficient) is approximately 2-3 mm, a depth that encompasses a large fraction of the entire distribution of renal nerves, as shown in FIG. 1B above.

Alternatively, 980 nm may heat more rapidly at lower powers with greater surface heating and selective absorption by hemoglobin and myoglobin. Laser heating in the wavelength band between about 1120 nm and about 1250 nm may be desirable in view of the low hemoglobin and myoglobin (Hb-O₂/MbO₂) absorption, selective absorption by lipids and proteins, and moderate water absorption in this band. Since the renal nerves located farthest from the lumen are mostly embedded in adipose tissue, and are themselves coated in lipid-rich myelin, the peak of lipid absorption at 1210 nm may promote deeper heat generation in close proximity to the nerves.

Thus, based on tissue heating properties and light source availability, the light source wavelengths may include about 980 nm, about 1060 nm, and about 1210 nm. The 980 nm wavelength allows the fastest heating, but with greater risk of superficial peri-neural tissue damage, 1210 nm may require longer treatment times, but would minimize superficial peri-neural tissue damage and 1210 nm may promote deeper heat generation.

Some suitable high power light sources include diode lasers and doped fiber lasers pumped with diode lasers. For renal denervation, the required incident laser power is estimated to be in the range of about 2 watts to 20 watts depending on the wavelength and dwell time. To achieve a temperature of 60-80° C. at a tissue depth of 1-2 mm, dwell time may be in the range of about 2 seconds to 20 seconds. Suitable light sources may include 980 nm diode lasers and 1060 nm Yb-doped fiber lasers with single-mode and multimode powers between about 10 watts and about 100 watts, which are available from IPG Photonics Corporation (Oxford, Mass.). Diode laser emitters with about 3-10 watts output in the 1208-1290 nm range are available from LDX Optronics, Inc or Innolume, Inc. Such light sources may be used to irradiate the arterial wall through catheters to laser ablate renal nerves within the renal artery as will be described in the embodiments below.

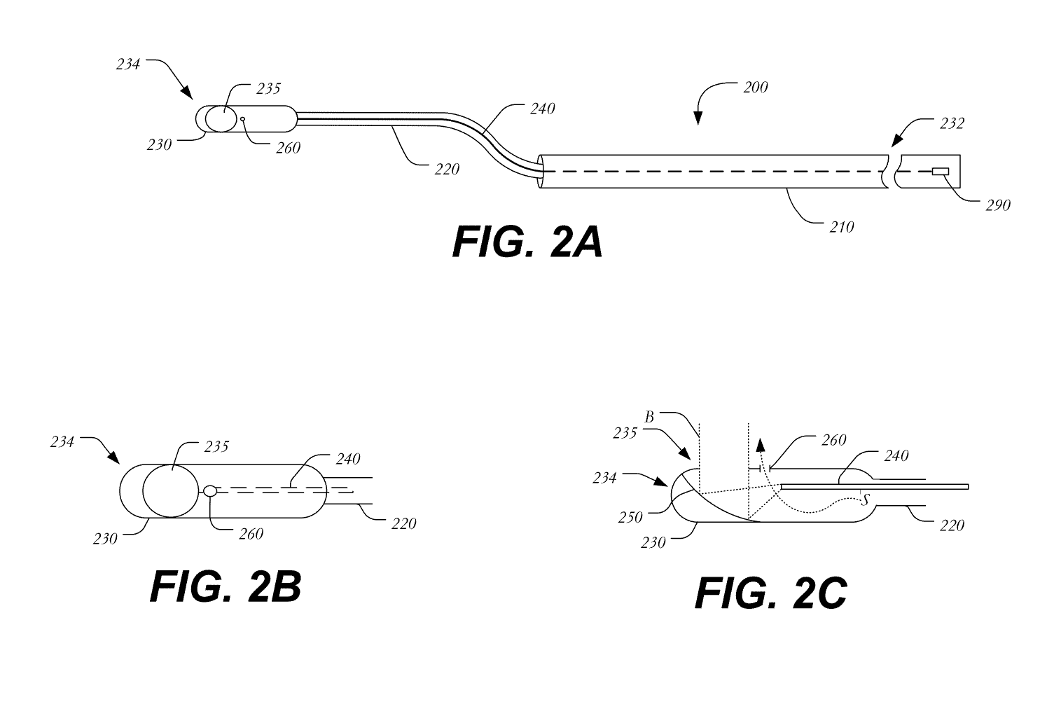

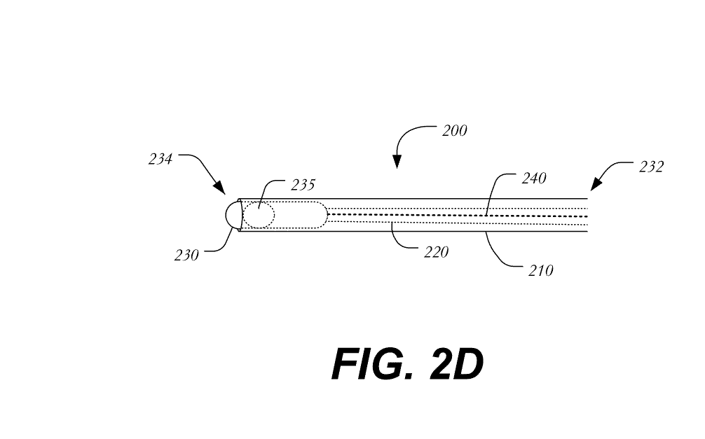

FIG. 2A is a side perspective view of a laser ablation system 200 in accordance with a first embodiment. Laser ablation system 200 extends between a leading end 234 and a trailing end 232 and includes a sheath 210, a tube 220 and a beam director 230.

Sheath 210 may be sized for transfemoral delivery into a patient's renal artery and may be formed of a substantially hollow tube. A pre-formed, steerable hollow tube 220 may be disposed within sheath 210. Tube 220 may be formed of nitinol or other shape-memory material. Tube 220 may have a circular or oval cross-sectional shape to allow rotation within sheath 210. Housed within tube 220 is an optical fiber 240 that is coupled to a light source 290 and extends to beam director 230 at the leading end 234 of the laser ablation system 200. Light source 290 may be selected from among any of the light sources described above or other light sources capable of ablating portions of tissue in the renal artery.

Located on one side of beam director 230 is an optical aperture 235 through which energy may be delivered to the target tissue. FIGS. 2B and 2C are enlarged top and side views of the leading end 234 of laser ablation system 200, illustrating beam director 230. As seen in FIG. 2C, a reflector 250, such as a flat or curved mirror, may be disposed within beam director 230 to direct a light beam B at the target tissue. Alternatively, a beam director consisting of scattering particles, such as microcrystalline titanium dioxide, can be employed to disperse the light from the fiber.

Optionally, a fluid for irrigation, such as saline, may be passed through tube 220. Moreover, beam director 230 may further include an optional irrigation window 260, near optical aperture 235, through which saline S may be delivered to the tissue to provide cooling and to flush away the thin residual blood layer between beam director 230 and the target vessel wall. Laser ablation system 200 may further include elements for reflectance or spectrophotometric feedback to indicate adequate tissue contact, which will be discussed below.

As seen in FIG. 2D, the optical elements, including tube 220, optical fiber 240 and beam director 230, may be retracted within sheath 210 for delivery into and removal from the patient's body. Additionally, beam director 230 may include a blunt tip at leading end 234 so as not to cause trauma to the patient's body during delivery.

In use, the laser ablation system 200 of FIGS. 2A-D may be introduced into the body in the retracted position shown in FIG. 2D using a transfemoral or other suitable approach. Laser ablation system 200, including sheath 210, may be advanced until the ostium of the renal artery. Optical fiber 240 and beam director 230 may be advanced out of sheath 210 and into the renal artery so that it is positioned at a point slightly proximal of the renal artery bifurcation. A controller (not shown) may be used to control power to a light source such as, for example, a diode laser, and a collimated laser beam for a predetermined period of time (e.g., 15 seconds). A transneural lesion is created across the renal nerves to disrupt nerve impulses traveling through the nerves. Tube 220 may then be rotated by, for example, 90 degrees so that a second lesion may be formed at the same position along the length of the renal artery. This process may be repeated as necessary so that four discrete lesions are formed at the same longitudinal position along the renal artery. Tube 220 may then be slightly retracted and the process repeated so that a second set of four discrete lesions may be formed. It will be understood that the number of lesions formed may include any number such as, for example, one, two, three, four, five, six or more lesions. Moreover, the lesions need not be formed at the same longitudinal position along the renal artery and may be formed in any suitable pattern. For example, helical, spiral or circular patterns of lesions may be formed as desired.

After forming the desired number of lesions in the renal artery, tube 220 and beam director 230 may be withdrawn into sheath 210 and the laser ablation system 200 may be retracted from the ostium of the renal artery. Laser ablation system 200 may then be repositioned in the ostium of the contralateral renal artery and the ablation process repeated in the second renal artery. When finished, tube 220 and beam director 230 may be retracted within sheath 210 and laser ablation system 200 may be removed from the patient's body.

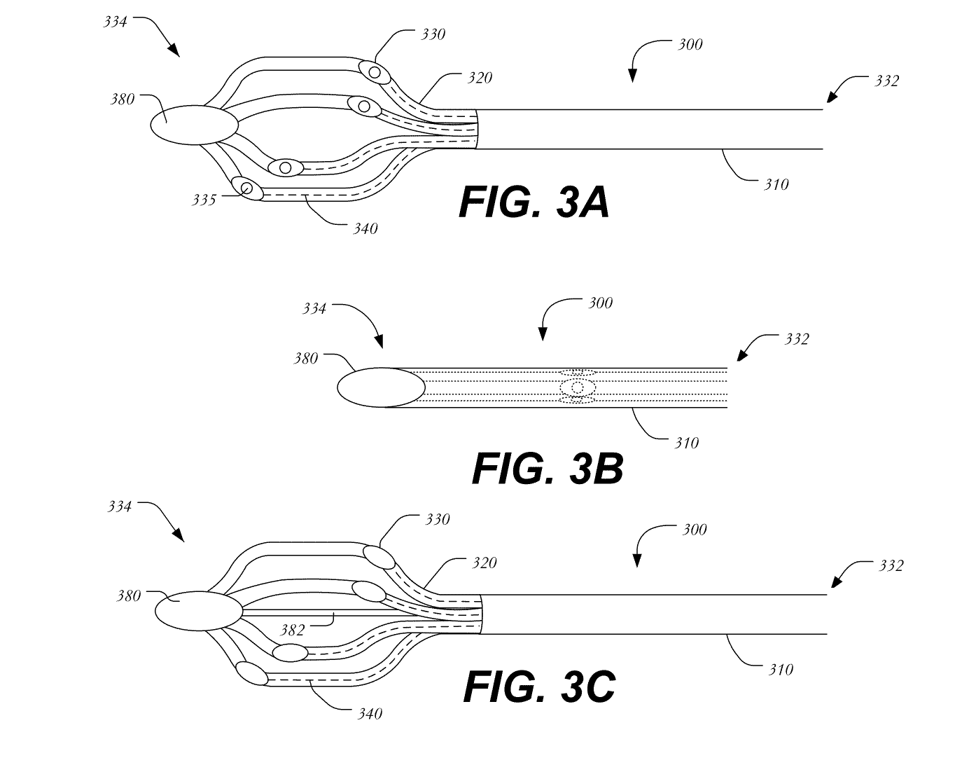

FIG. 3A is a side view of a laser ablation system 300 in accordance with a second embodiment of the present invention. Laser ablation system 300 extends between a leading end 334 and a trailing end 332, and includes a sheath 310, a series of tubes 320 and a series of beam directors 330.

The tubes 320 form a collapsible basket-like construction, each tube 320 housing an independent optical fiber 340 for delivering light energy to target tissue. Tubes 320 may be resiliently biased such that, when advanced out from sheath 310, the tubes 320 radially expand as shown in FIG. 3A. Alternatively, the tubes 320 may be coupled to a wire actuated through a handle that expands and collapses the tubes 320. In certain embodiments, the tubes 320 may be retracted within sheath 310 during delivery and retrieval, as shown in FIG. 3B.

While FIG. 3A illustrates a laser ablation system having four tubes 320 arranged circumferentially apart by 90 degrees, it will be understood that any number of tubes 320 and optical fibers 340 may be used in a laser ablation system. Moreover, each of the tubes 320 may independently connect to different light sources and may be configured to deliver energy of different wavelengths or dwell times. Alternatively, tubes 320 may all be connected to the same light source and configured to deliver energy of the same wavelength for the same duration. Energy may be delivered through tubes 320 sequentially or at the same time.

As seen in FIG. 3A, each tube 320 may be further connected to an individual beam director 330, each beam director having an optical aperture 335 and a reflector (not shown) to deliver a laser beam to the target tissue. Each of the tubes 320 may be connected to a single distal expander 380 located at leading end 334 to prevent the concentration of optical energy at the tip of the laser ablation system. In one variation, seen in FIG. 3C, a wire 382 may connect to distal expander 380 and pass through sheath 310 to a handle (not shown). Wire 382 may be used to expand and collapse the basket-like construction without requiring that all of the tubes be retracted within sheath 310.

Laser ablation system 300 may be used in a manner similar to laser ablation system 200, except that multiple ablations may be performed simultaneously or sequentially using the individual beam directors 330 at a single longitudinal position along the length of the artery without having to rotate the laser ablation system or the tubes 320. For example, four lesions may be made at a first longitudinal position along the renal artery. The tubes 320 may then be retracted slightly and rotated to make that a second set of lesions at a second longitudinal position along the renal artery such that the second set of lesions do not radially align with the first set of lesions. The laser ablation system 300 may then be retracted and the process repeated in the contralateral renal artery.

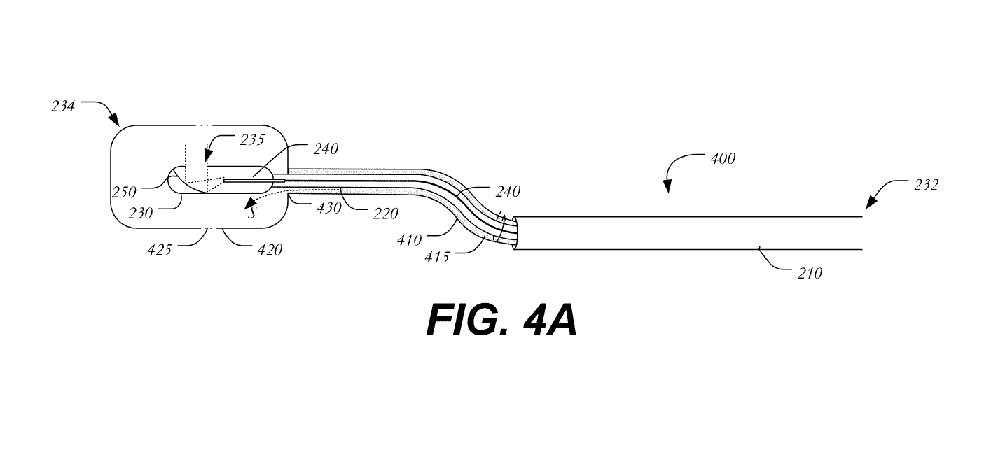

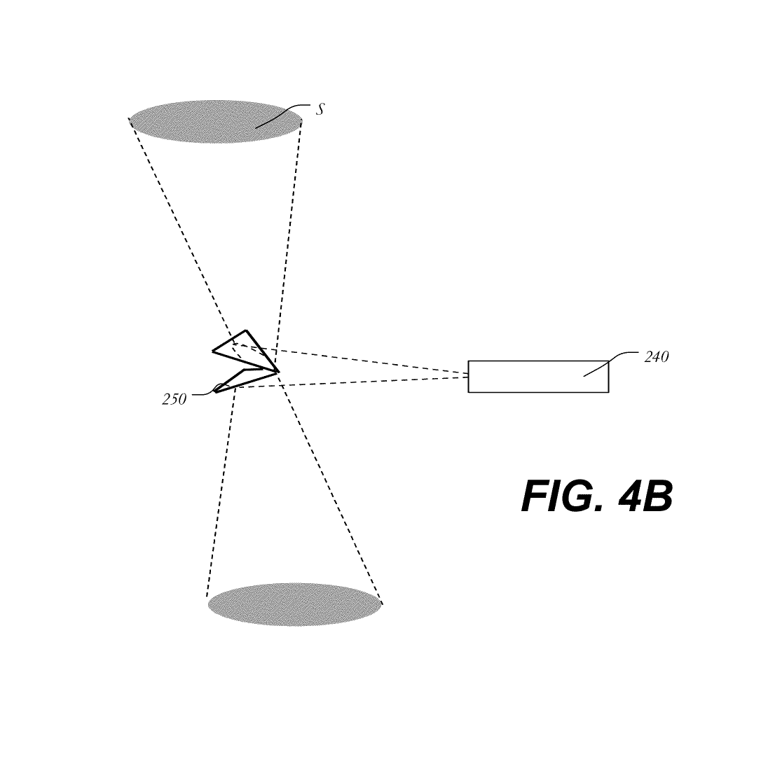

FIG. 4A is a side perspective view of a laser ablation system 400 in accordance with a third embodiment of the present invention. Laser ablation system 400 incorporates many of the same components of laser ablation system 200, including a sheath 210, a tube 220 housing an optical fiber 240 and a beam director 230 having a reflector 250 and a window 235. Laser ablation system 400 further includes a polymeric inflation shaft 410 surrounding tube 220 and defining an interior lumen 415 for delivering an inflation medium, such as heavy water saline, D₂O saline or CO₂, via port 430 to a balloon 420. Balloon 420 may be useful in centering the laser ablation system 400 during therapy.

Balloon 420 may be a compliant low-pressure balloon capable of expanding when an inflation medium, such as saline, is introduced therein. Though FIG. 4A illustrates a substantially rectangular balloon 420, it will be understood that balloon 420 may be formed with any desirable transverse cross-sectional shape including circular, oval, square or other suitable shape. Balloon 420 may be elongated such that laser ablation system 400 is able to form a set of lesions at a first longitudinal position in the renal artery, be pulled back within the balloon, and form a second set of lesions at a different longitudinal position in the renal artery. Balloon 420 may also have a radius that is large enough to allow rotation of the beam director 230 within the body of the balloon. Thus, a large enough balloon will reduce the need for multiple repositioning operations of the balloon 420 and sheath 210. Instead, tube 220 may be manipulated and beam director 230 may be translated and rotated within stationary balloon 420 to form ablations at different tissue sites. It should be understood that FIG. 4A illustrates a balloon 420 in the inflated configuration and that when deflated, balloon 420 may be small enough to be retracted within sheath 210.

Balloon 420 may include a plurality of perforations 425 around its circumference to allow flushing of thin residual blood from between the balloon and the vessel wall. Perforations 425 may also be useful to allow cooling of tissue. When saline is used as the medium for inflating balloon 420, the same saline may also be used to provide flushing and/or cooling.

In embodiments where a balloon is used, feedback may be optional. This may include feedback to detect fiber breakage, which may be accomplished by detecting a sudden increase in the light reflected from the fiber caused by specular reflection at the broken fiber interface. Light from a broken fiber can be distinguished from light scattered diffusely from blood or tissue by its nearly flat wavelength dependence. Moreover, the radius of curvature of a faceted reflector may be used to form an elliptical beam with a longer spot size along the longitudinal dimension of the laser ablation system as seen by spot size “S” of FIG. 4B. Using this variation, a quasi-circumferential lesion pattern may be formed so that only a single circumferential ablation is needed, thereby eliminating the need for pullback to ablate tissue at multiple longitudinal positions.

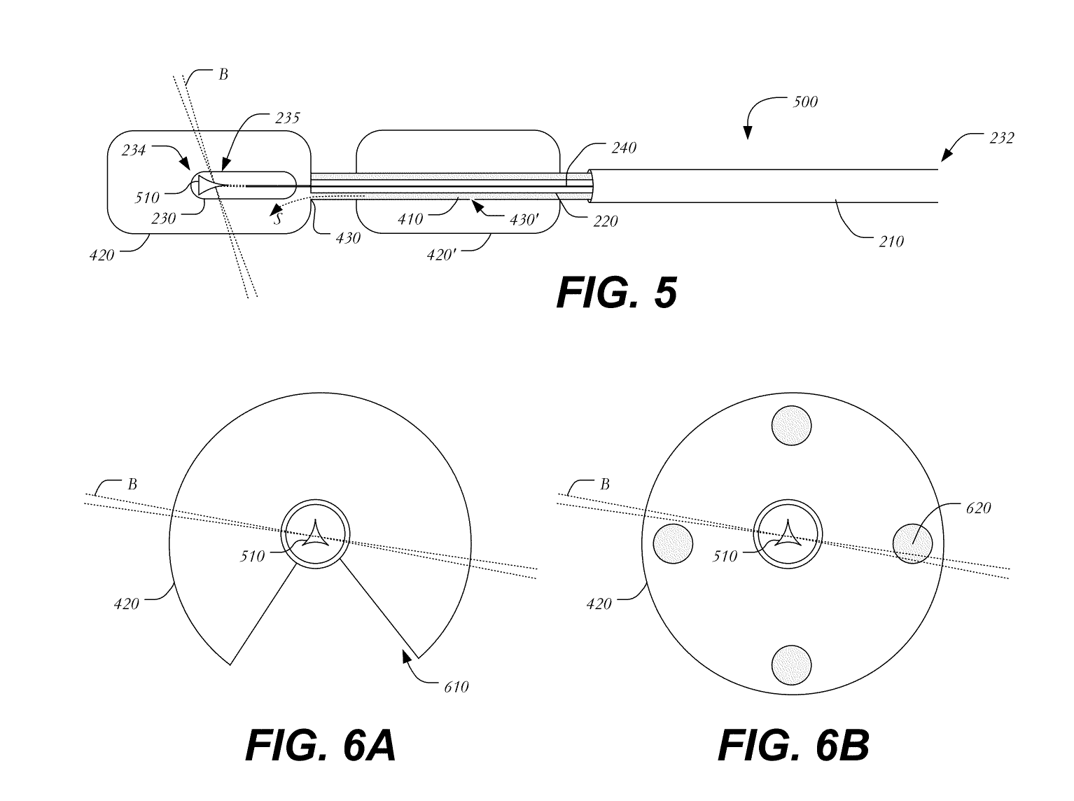

FIG. 5 is a side perspective view of a laser ablation system 500 in accordance with a fourth embodiment of the present invention. Laser ablation system 500 incorporates many of the components described above and includes a second balloon 420′. Balloon 420′ may be sized and shaped similarly to the first balloon 420. Balloon 420′ may be useful in improving the centering performance of laser ablation system 500 within the renal artery. Balloons 420, 420′ may be filled with an inflation medium via ports 430, 430′, respectively. Balloons 420, 420′ may be in fluid communication with a single inflation shaft 410 and may be inflated sequentially (e.g., saline is delivered through shaft 410 and balloon 420 begins to fill after balloon 420′ fills completely).

A faceted reflector 510 may be disposed within beam director 230 and configured to focus laser beams B in two or more directions as shown in FIG. 5. Faceted reflector 510 may eliminate the need for rotating tube 220 or optical fiber 240 and allows the laser ablation system 500 to form multiple lesions at the same longitudinal position, reducing the complexity of the procedure and reducing the amount of time spent rotating the laser ablation system. It is noted that faceted reflector 510 may be capable of ablating three, four, five or more locations at the same longitudinal position. Alternatively, multiple fibers 240 may be used within each of the balloons to eliminate the need for pulling back the laser ablation system to form lesions at a second longitudinal position. It may also be possible to ablate tissue through the balloon.

In one variation of the balloons discussed above, a balloon may include features for allowing the flow of blood therethrough. FIG. 6A is a schematic transverse cross-sectional view of a balloon 420 having a channel 610 extending along the entire length thereof. Channel 610 may be formed as a triangular cross-sectional cutout. Channel 610 may allow continuous blood flow through the renal artery during ablation. Accordingly, balloon 420 serves dual purposes. First, continuous blood flow past the balloon may be beneficial in providing cooling to the ablated tissue. Second, channels 610 maintain the supply of blood to the kidney, which prevents disruption of physiological activity. It will be understood that multiple channels 610 may be formed at spaced positions around the perimeter of balloon 420 and that the shapes of the channels may be modified. For example, channels 610 may be formed as lumens 620 extending longitudinally through the interior of the balloon, as shown in FIG. 6B.

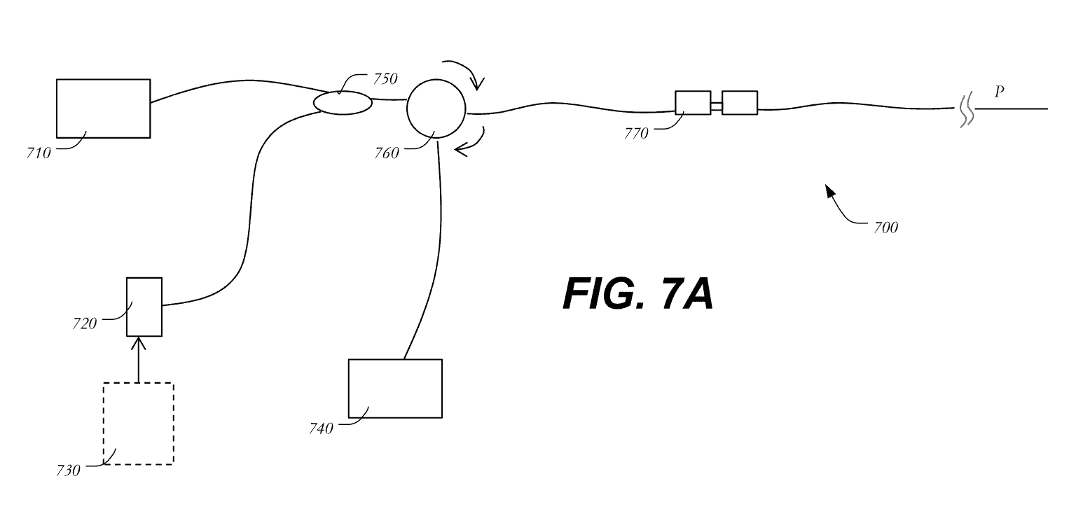

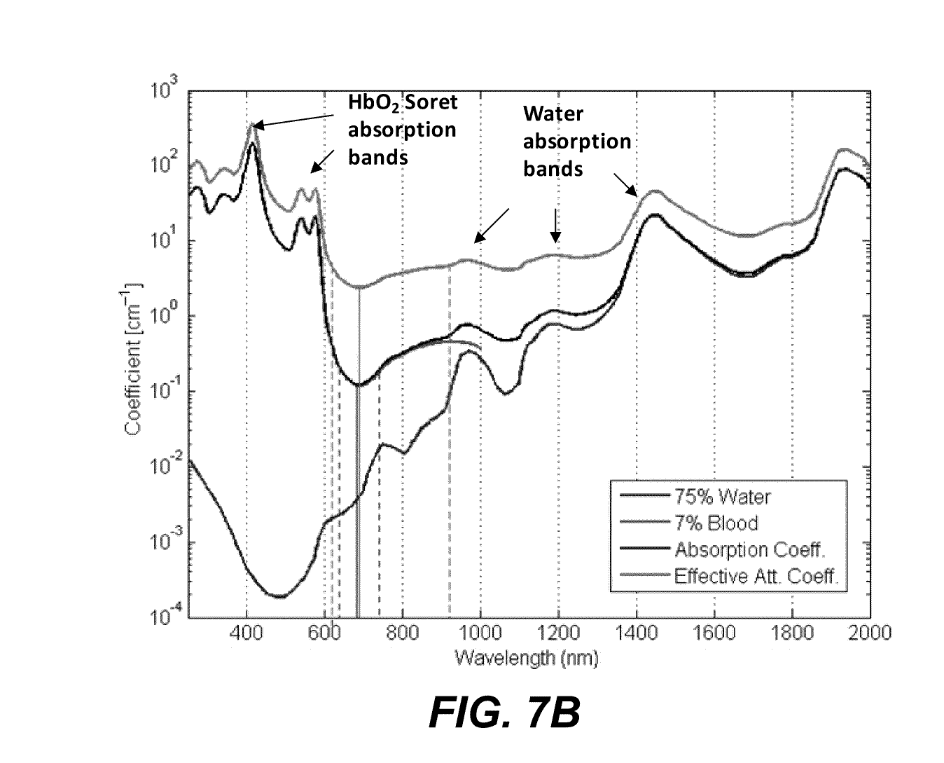

Various feedback control methods may be used during the ablation procedure to ensure proper therapy. In one embodiment, the same optical fibers used for laser irradiation of the tissue may be used to sense reflectance from tissue in the path of the beam. Additionally, broadband illumination and detection with a spectrometer through a 2×1 coupler or wavelength-division multiplexer may provide the most detailed information about the content of blood, water, and other substances in the path of the beam. As seen in FIG. 7A, an optical spectrum feedback system 700 may include a first high-power laser emitter 710 for supplying energy at an ablation wavelength to beam director “P” of the laser ablation system, a low-power laser or broadband light source 720 for emitting energy within a band of interrogation wavelengths and an optional modulator 730. The two lasers 710,720 connect to an optical coupler or wavelength-division multiplexer 750, which connects to optical connector 770 via circulator 760. If the circulator 760 cannot pass the emission wavelengths of both the ablation laser and interrogation light source, the circulator 760 can be replaced with a wavelength division multiplexer or broadband optical coupler. A detector 740 such as a single photodetector, a spectrometer or other photometric apparatus for measuring light intensity within selected wavelength bands is connected to circulator 760 to receive the backscattered light from the tissue through the optical fiber in the ablation catheter.

With this configuration, the Soret Bands, intense peaks in the blue and green regions of the oxygenated hemoglobin (HbO₂) absorption spectrum, may serve as a unique spectral feature of blood as seen in FIG. 7B. Reflectance measured in spectral bands at which water absorbs significantly (e.g., 900-1000 nm, 1105-1250 nm or 1350-1550 nm) may serve as a variable for monitoring tissue contact and hydration both before and after irradiation. Moreover, fiber breakage and blood clearance may also be detected using a simpler, less expensive optical system based on modulated illumination with one or two low-power diodes and detection with a single detector.

Although the system and method herein have been described with reference to particular embodiments, it is to be understood that these embodiments are merely illustrative of the principles and applications of the present system and method. It is therefore to be understood that numerous modifications may be made to the illustrative embodiments and that other arrangements may be devised without departing from the spirit and scope of the present system and method as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. An ablation catheter comprising:
an elongated sheath configured for intravascular usage;
an inner tube disposed within the elongated sheath, the inner tube being rotatable and translatable relative to the sheath;
an optical fiber having a proximal end and a distal end, the optical fiber being disposed within the inner tube and extending longitudinally therethrough, the proximal end of the optical fiber being optically coupleable to a light source;
a beam director coupled to the distal end of the optical fiber; and
a controller configured to focus energy from the light source through the beam director on target tissue of a blood vessel to ablate the target tissue at a depth of 0.5 mm to 2.5 mm from an inner wall of the blood vessel.

2. The ablation catheter of claim 1, wherein the light source is selected from the group consisting of a diode laser and a doped fiber laser pumped with a diode laser.

3. The ablation catheter of claim 1, further comprising a reflector disposed within the beam director and configured to focus the energy from the light source on the target tissue.

4. The ablation catheter of claim 1, wherein the light source emits light at a wavelength of between 950 nm and 1300 nm.

5. The ablation catheter of claim 1, wherein the controller is configured to control the emission of light from the light source for a duration of between 2 seconds and 20 seconds.

6. The ablation catheter of claim 1, further comprising a plurality of irrigation fluid channels on the beam director and configured to direct irrigation fluid toward the target tissue.

7. The ablation catheter of claim 1, further comprising a centering balloon coupled to an inflation shaft, the centering balloon being configured to properly position the ablation catheter within the blood vessel when an inflation medium is introduced through the inflation shaft to inflate the centering balloon.

8. The ablation catheter of claim 7, wherein the centering balloon is disposed about the beam director.

9. The ablation catheter of claim 7, wherein the centering balloon is elongated to allow the beam director to form discrete lesions at multiple longitudinal levels along the blood vessel within the centering balloon.

10. The ablation catheter of claim 7, wherein the centering balloon includes at least one channel extending from a first longitudinal end of the balloon to a second longitudinal end of the balloon to allow blood to continuously pass through the blood vessel while the catheter is disposed therein.

11. The ablation catheter of claim 7, wherein the centering balloon includes at least one perforation to allow the inflation medium to pass therethrough into the blood vessel.

12. The ablation catheter of claim 7, wherein the inflation medium is saline.

13. The ablation catheter of claim 1, further comprising a plurality of centering balloons coupled to an inflation shaft, the plurality of centering balloons being configured to properly position the ablation catheter within a blood vessel when an inflation medium is introduced through the inflation shaft to inflate the centering balloons.

14. The ablation catheter of claim 1, further comprising a faceted reflector disposed within the beam director and configured to focus the energy from the light source in at least two different radial directions.

15. The ablation catheter of claim 1, further comprising a detector disposed within the beam director and configured to provide feedback of energy reflected from the target tissue.

16. The ablation catheter of claim 1, wherein the optical fiber is configured and arranged to sense optical feedback.

17. An ablation catheter comprising:
an elongated sheath configured for intravascular usage;
a plurality of tubes disposed within the elongated sheath and translatable relative to the elongated sheath, the plurality of tubes being resiliently biased outwardly away from the elongated sheath;
an optical fiber disposed within each of the tubes, each of the optical fibers having a proximal end and a distal end, the proximal end of each of the optical fibers being optically coupleable to a first light source;
a plurality of beam directors coupled to each of the optical fibers; and
a controller configured to focus energy from the first light source through each of the plurality of beam directors on target tissues of a blood vessel to ablate the target tissues at depths of 0.5 mm to 2.5 mm from an inner wall of the blood vessel.

18. The ablation catheter of claim 17, wherein the plurality of tubes form a collapsible basket-like arrangement.

19. The ablation catheter of claim 17, wherein the plurality of tubes are formed of nitinol.

20. The ablation catheter of claim 17, wherein the plurality of tubes comprises four tubes arranged circumferentially apart by 90 degrees.

21. The ablation catheter of claim 17, wherein the optical fibers in each of the plurality of tubes deliver energy of the same wavelength.

22. The ablation catheter of claim 17, wherein the optical fibers are configured and arranged to sense optical feedback.

23. The ablation catheter of claim 17, further comprising a detector configured to measure light intensity from optical feedback.

24. The ablation catheter of claim 17, further comprising a spectrometer configured to measure light intensity within predetermined wavelength bands.

25. The ablation catheter of claim 17, further comprising a second light source and an optical coupler.
